(19) United States
(12) Patent Application Publication
(10) Pub. No.: US 2014/0242582 A1
(43) Pub. Date: Aug. 28, 2014

(54) DETECTION OF GENETIC ABNORMALITIES USING LIGATION-BASED DETECTION AND DIGITAL PCR

(71) Applicant: Ariosa Diagnostics, Inc., San Jose, CA (US)

(72) Inventors: Arnold Oliphant, San Jose, CA (US); Jacob Zahn, San Jose, CA (US)

(73) Assignee: Ariosa Diagnostics, Inc., San Jose, CA (US)

(21) Appl. No.: 14/183,150

(22) Filed: Feb. 18, 2014

Related U.S. Application Data
(60) Provisional application No. 61/770,678, filed on Feb. 28, 2013.

(51) Int. Cl. C12Q 1/68

(52) U.S. Cl. CPC C12Q 1/6834
USPC 435/6.11

(57) ABSTRACT

The present invention provides assays systems and methods for detection of genetic variants in a sample, including copy number variation and single nucleotide polymorphisms. The invention preferably employs the technique of tandem ligation, i.e. the ligation of two or more fixed sequence oligonucleotides and one or more bridging oligonucleotides complementary to a region between the fixed sequence oligonucleotides combined with digital PCR detection.

### CROSS-REFERENCE TO RELATED APPLICATIONS

This application claims the benefit of U.S. Provisional Patent Application Ser. No. 61/770,678, filed Feb. 28, 2013 and is assigned to the assignee of the present application and incorporated herein by reference.

### FIELD OF THE INVENTION

This invention relates to multiplexed selection, amplification, and detection using digital PCR for selected genomic regions from a sample.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Genetic abnormalities account for a wide number of pathologies, including pathologies caused by chromosomal aneuploidy (e.g., Down syndrome), germline mutations in specific genes (e.g., sickle cell anemia), and pathologies caused by somatic mutations (e.g., cancer). Diagnostic methods for determining such genetic anomalies have become standard techniques for identifying specific diseases and disorders, as well as providing valuable information on disease source and treatment options.

Copy-number variations are alterations of genomic DNA that correspond to relatively large regions of the genome that have been deleted or amplified on certain chromosomes. CNVs can be caused by genomic rearrangements such as deletions, duplications, inversions, and translocations. Copy number variation has been associated with various forms of cancer (Cappuzzo F, Hirsch, et al. (2005) 97 (9): 643-655) neurological disorders (Sebat, J., et al. (2007) Science 316 (5823): 445-9, including autism (Sebat, J., et al. (2007) Science 316 (5823): 445-9), and schizophrenia (St Clair, D., (2008) Schizophr Bull 35 (1): 9-12). Detection of copy number variants of a chromosome of interest or a portion thereof in a specific cell population can be a powerful tool to identify genetic diagnostic or prognostic indicators of a disease or disorder.

Detection of copy number variation is also useful in detecting chromosomal aneuploidies in fetal DNA. Conventional methods of prenatal diagnostic testing currently requires removal of a sample of fetal cells directly from the uterus for genetic analysis, using either chorionic villus sampling (CVS) between 11 and 14 weeks gestation or amniocentesis after 15 weeks. However, these invasive procedures carry a risk of miscarriage of around 1% (Mujezinovic and Alfirevic, Obstet Gynecol 2007:110:687-694), A reliable and convenient method for non-invasive prenatal diagnosis has long been sought to reduce this risk of miscarriage and allow earlier testing.

Single nucleotide polymorphisms (SNPs) are single nucleotide differences at specific regions of the genome. The average human genome typically has more than three million SNPs when compared to a reference genome. SNPs have been associated with various diseases, including cancer, cardiovascular disease, cystic fibrosis, and diabetes. Detection of SNPs can be a powerful tool to identify genetic diagnostic or prognostic indicators of a disease or disorder. It is often desirable to detect many different SNPs in the same sample.

There is a need for methods of screening for copy number variations that employs an efficient, reproducible yet non-invasive detection system. The present invention addresses this need.

### SUMMARY OF THE INVENTION

This Summary is provided to introduce a selection of concepts in a simplified form that are further described below in the Detailed Description. This Summary is not intended to identify key or essential features of the claimed subject matter, nor is it intended to be used to limit the scope of the claimed subject matter. Other features, details, utilities, and advantages of the claimed subject matter will be apparent from the following written Detailed Description including those aspects illustrated in the accompanying drawings and defined in the appended claims.

The present invention provides assay systems and methods for detection of genetic copy number variations, polymorphisms, and mutations. The invention employs the technique of selecting genomic regions for analysis using fixed sequence oligonucleotides, followed by detection of the selected genomic regions using digital PCR techniques to determine the frequency of the selected genomic regions of interest in a sample. The fixed sequence oligonucleotides hybridize to selected genomic regions of interest, and are joined via ligation and/or extension to create a ligation product.

In a preferred aspect, the fixed sequence oligonucleotides are used with one or more bridging oligonucleotides which hybridize between the two fixed sequence oligonucleotides. The fixed sequence oligonucleotides and bridging oligonucleotide are joined by ligation—i.e. the ligation of the fixed sequence oligonucleotides and the one or more bridging oligonucleotides—to form a ligation product. The bridging oligonucleotides can hybridize in the genomic region between and immediately adjacent to the fixed sequence oligonucleotides, or may bind to a non-adjacent region between the two fixed sequence oligonucleotides and one of the fixed sequence oligonucleotides and/or the one or more bridging oligonucleotides are extended so that the fixed sequence oligonucleotides and the one or more bridging oligonucleotides are hybridized contiguously for subsequent ligation. The ligation products are further amplified, e.g., using primer sequences available on one or both of the fixed sequence oligonucleotides, to create amplification products. Preferably, the primer sequences used are universal primer sequences common to multiple amplification products.

In certain aspects, the amplification products comprise indices that facilitate detection of the selected genomic regions of interest. For example, in certain aspects, each amplification product corresponding to a selected genomic region comprises a chromosomal index that corresponds to the chromosome on which the selected genomic region is known to be located. The chromosomal indices are then detected in some embodiments using digital PCR. In certain aspects, the amplification products comprise chromosomal indices which are analyzed using fluorescently labeled primers, wherein in preferred embodiments different fluorescent labels are used with different chromosomal indices.

Preferred digital PCR methods involve partitioning of diluted amplification products into a plurality of discrete test sites such that most of the discrete test sites comprise either zero or one amplification product. The amplification products are then analyzed to provide a representation of the frequency of the selected genomic regions of interest in the sample. Analysis of one amplification product per discrete test site results in a binary “yes-or-no” result for each discrete test site, allowing the selected genomic regions of interest to be quantified and the relative frequency of the selected genomic regions of interest in relation to one another be determined. In certain aspects, in addition or as an alternative to analysis of whole chromosomes, multiple analyses may be performed using amplification products corresponding to genomic regions from predetermined subchromosomal regions, where analysis is carried out for two or more predetermined subchromosomal regions from the same chromosome. Results from the analysis of two or more predetermined subchromosomal regions of the chromosome are used to quantify and determine the relative frequency of the number of amplification products associated with the chromosome. Using two or more predetermined subchromosomal regions to determine the frequency of a particular chromosome in a sample reduces a possibility of bias through, e.g., variations in amplification efficiency, which may not be readily apparent if a chromosome is quantified through a single detection assay. Quantification of subchromosomal regions also allows identification of partial chromosomal aneuploidies or other copy number variations (e.g., large insertions or deletions) which do not affect the entire chromosome.

In one general aspect, the invention provides methods for detecting a frequency of selected genomic regions of interest in a sample, comprising the steps of providing a sample comprising major and minor source cell-free DNA; introducing at least two sets of first and second fixed sequence oligonucleotides to the sample under conditions that allow each set of fixed sequence oligonucleotides to specifically hybridize to different genomic regions of interest; performing a ligation step to create ligation products; amplifying the ligation products to create amplification products that reflect a relative frequency of the genomic regions of interest in the sample; partitioning the amplification products into a plurality of discrete test sites such that the plurality of discrete test sites comprises either one or zero of the amplification products; and analyzing the amplification products in the plurality of discrete test sites to provide a representation of the frequency of the genomic regions of interest in the sample.

As described above, the invention in some embodiments employs a tandem ligation, e.g., the ligation of two or more non-adjacent, fixed sequence oligonucleotides and a bridging oligonucleotide that is complementary to a region between and directly adjacent to the portion of the genomic region of interest complementary to the fixed sequence oligonucleotides.

In one general aspect, the invention provides an assay system for determining a frequency of selected genomic regions of interest in a sample, comprising the steps of: providing a sample comprising major and minor source cell-free DNA; introducing at least two sets of first and second fixed sequence oligonucleotides to the sample under conditions that allow each set of fixed sequence oligonucleotides to specifically hybridize to different genomic regions of interest; introducing one or more bridging oligonucleotides for each set of fixed sequence oligonucleotides under conditions that allow the bridging oligonucleotides to specifically hybridize to complementary regions in the genomic regions of interest, wherein the one or more bridging oligonucleotide is complementary to a region between the first and second fixed sequence oligonucleotides of the sets; performing a ligation step to create ligation products; amplifying the ligation products to create amplification products that reflect a relative frequency of the genomic regions of interest in the sample; partitioning the amplification products into a plurality of discrete test sites such that the plurality of discrete test sites comprises either one or zero of the amplification products; and analyzing the amplification products in the plurality of discrete test sites to provide a representation of the frequency of genomic regions of interest in the sample.

In certain aspects, the two sets of first and second oligonucleotides may be introduced to the samples, and the region between the first and second fixed sequence oligonucleotides of each set may be extended with a polymerase and dNTPs to create adjacently hybridized fixed sequence oligonucleotides. Ligation of the two fixed sequence oligonucleotides in each set may then be carried out to create a ligation product complementary to the first and second genomic regions of interest.

In one general aspect, the invention provides a method for determining a frequency of selected genomic regions of interest in a sample, comprising the steps of: providing a sample comprising major and minor source cell-free DNA; introducing at least two sets of first and second fixed sequence oligonucleotides to the sample under conditions that allow each set of fixed sequence oligonucleotides to hybridize to different selected genomic regions of interest; extending the region between the first and second fixed sequence oligonucleotides of the sets with a polymerase and dNTPs to create adjacently hybridized fixed sequence oligonucleotides; performing a ligation step to create ligation products; amplifying the ligation products to create amplification products that reflect a relative frequency of the genomic regions of interest in the sample; partitioning the amplification products into a plurality of discrete test sites such that the plurality of discrete test sites comprises either one or zero of the amplification products; and analyzing the amplification products in the plurality of discrete test sites to provide a representation of the frequency of genomic regions of interest in the sample.

In yet another general aspect, the invention provides a method for determining a frequency of selected genomic regions of interest in a sample, comprising the steps of: providing a sample comprising major and minor source cell-free DNA; introducing at least two sets of first and second fixed sequence oligonucleotides to the sample under conditions that allow each set of fixed sequence oligonucleotides to hybridize to different yet adjacent selected genomic regions of interest; performing a ligation step to create ligation products; amplifying the ligation products to create amplification products that reflect a relative frequency of the genomic regions of interest in the sample; partitioning the amplification products into a plurality of discrete test sites such that the plurality of discrete test sites comprises either one or zero of the amplification products; and analyzing the amplification products in the plurality of discrete test sites to provide a representation of the frequency of genomic regions of interest in the sample.

In yet an additional exemplary embodiment, the present invention provides a method for detecting nucleic acid regions of interest in a genetic sample, comprising the steps of: providing a genetic sample; introducing at least two fixed sequence oligonucleotides to the genetic sample under conditions that allow the fixed sequence oligonucleotides to specifically hybridize to complementary regions in each nucleic acid region of interest, wherein both ends of each fixed sequence oligonucleotide are complementary to a single nucleic acid region of interest, and wherein upon hybridization each fixed sequence oligonucleotide forms a pre-circle oligonucleotide; introducing one or more bridging oligonucleotides to the genetic sample under conditions that allow the one or more bridging oligonucleotides to specifically hybridize to complementary regions in the nucleic acid regions of interest, wherein the one or more bridging oligonucleotides are complementary to a region between the region of the nucleic acid region of interest complementary to the ends of the fixed sequence oligonucleotides, and wherein the one or more bridging oligonucleotides hybridize contiguously between the ends of the fixed sequence oligonucleotides; ligating the hybridized oligonucleotides to create circular ligation products, a portion of which is complementary to the nucleic acid region of interest; amplifying the circular ligation product to create amplification products that reflect the relative frequency of the nucleic acid regions of interest in the genetic sample; partitioning the amplification products into a plurality of discrete test sites such that the plurality of discrete test sites comprises either one or zero of the amplification products; and analyzing the amplification products in the plurality of discrete test sites to provide a representation of the frequency of genomic regions of interest in the sample. In some aspects of this embodiment bridging oligonucleotides are not used and the pre-circle oligonucleotides are extended with dNTPs and a polymerase before ligation. In yet other aspects of this embodiment, the ends of the pre-circle oligonucleotides hybridize adjacent to one another, and neither a bridging oligonucleotide nor an extension reaction is needed before ligation.

In certain aspects, at least one of two fixed sequence oligonucleotides used in the assay system preferably comprises a universal primer region that is used in amplification of the ligation product. Alternatively, the universal primer sequence can be added to the ligation products following the ligation of the hybridized fixed sequence—and bridging oligonucleotides, if present—e.g., through ligation of adapters comprisins.

In one aspect of the invention, the sets of first and second fixed sequence oligonucleotides are introduced to the sample and specifically hybridized to complementary portions of the selected genomic regions of interest prior to introduction of the bridging oligonucleotides. In such an aspect, the sets of first and second fixed sequence oligonucleotides and the selected genomic regions to which they are hybridized are optionally isolated following the hybridization of the sets of fixed sequence oligonucleotides to remove any excess unbound fixed sequence oligonucleotides in the reaction prior to the introduction of the bridging oligonucleotides.

Alternatively, the bridging oligonucleotides are introduced to the sample at the same time the sets of fixed sequence oligonucleotides are introduced, and are allowed to hybridize to the selected genomic region of interest.

The relative frequency of the selected genomic regions in the sample can be used to quantitate a chromosome or subchromosomal region which allows, e.g., determining chromosomal imbalances in a maternal sample due to aneuploidy in the fetus.

These aspects and other features and advantages of the invention are described in more detail below.

### BRIEF DESCRIPTION OF THE FIGURES

FIG. 1 is a simplified flow chart of the general steps for determining the frequency of selected genomic regions of interest in a sample.

FIG. 2 illustrates a general schematic for a ligation-based assay system of the invention.

FIG. 3 illustrates an assay system for detection of genomic regions of interest in accordance with certain aspects.

FIG. 4 illustrates an assay system for detection of genomic regions of interest in accordance with certain aspects.

FIG. 5 illustrates general steps for digital PCR detection of amplification products.

FIG. 6 illustrates an assay system for detection of genomic regions of interest on two different chromosomes.

FIG. 7 illustrates an assay system for detection of two or more alleles within a genomic region of interest.

### DEFINITIONS

The terms used herein are intended to have the plain and ordinary meaning as understood by those of ordinary skill in the art. The following definitions are intended to aid the reader in understanding the present invention, but are not intended to vary or otherwise limit the meaning of such terms unless specifically indicated.

The term “allele index” refers generally to a series of nucleotides that corresponds to a specific SNP. The allele index may contain additional nucleotides that allow for the detection of deletion, substitution, or insertion of one or more bases. The index may be combined with any other index to create one index that provides information for two properties (e.g., sample-identification index, allele-locus index).

The term “binding pair” means any two molecules that specifically bind to one another using covalent and/or non-covalent binding, and which can be used for attachment of genetic material to a substrate. Examples include, but are not limited to, ligands and their protein binding partners, e.g., biotin and avidin, biotin and streptavidin, an antibody and its particular epitope, and the like.

The term “chromosomal abnormality” refers to any genetic variant for all or part of a chromosome. The genetic variants may include but not be limited to any copy number variant such as duplications or deletions, translocations, inversions, and mutations.

The term “chromosomal index” refers generally to a series of nucleotides that correspond to a given chromosome. In a preferred aspect, the chromosomal index is long enough to uniquely identify an amplification product as being from a particular chromosome or predetermined subchromosomal region thereof. The chromosomal index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay.

The term “chromosome of interest” refers generally to a chromosome that is commonly associated with a copy number variation such as aneuploidy.

The terms “complementary” or “complementarity” are used in reference to nucleic acid molecules (i.e., a sequence of nucleotides) that are related by base-pairing rules. Complementary nucleotides are, generally, A and T (or A and U), or C and G. Two single stranded RNA or DNA molecules are said to be substantially complementary when the nucleotides of one strand, optimally aligned and with appropriate nucleotide insertions or deletions, pair with at least about 90% to about 95% complementarity, and more preferably from about 98% to about 100% complementarity, and even more preferably with 100% complementarity. Alternatively, substantial complementarity exists when an RNA or DNA strand will hybridize under selective hybridization conditions to its complement. Selective hybridization conditions include, but are not limited to, stringent hybridization conditions. Stringent hybridization conditions will typically include salt concentrations of less than about 1 M, more usually less than about 500 mM and preferably less than about 200 mM. Hybridization temperatures are generally at least about 2° C. to about 6° C. lower than melting temperatures (Tₘ).

The term “correction index” refers to an index that may contain additional nucleotides that allow for identification and correction of amplification, sequencing or other experimental errors including the detection of deletion, substitution, or insertion of one or more bases during sequencing as well as nucleotide changes that may occur outside of sequencing such as oligo synthesis, amplification, and any other aspect of the assay.

The term “diagnostic tool” as used herein refers to any composition or assay of the invention used in combination as, for example, in a system in order to carry out a diagnostic test or assay on a patient sample.

The term “sample” refers to any sample comprising all or a portion of the genetic information of an animal, and in particular a mammal. The sample may be used in its original the form, or may comprise nucleic acids isolated from a fluid or tissue of the animal. Preferably, the sample comprises blood, plasma or serum. In certain aspects, the sample comprises nucleic acids isolated from blood, plasma or serum, e.g., cellular or cell-free DNA

The term “hybridization” generally means the reaction by which the pairing of complementary strands of nucleic acid occurs. DNA is usually double-stranded, and when the strands are separated they will re-hybridize under the appropriate conditions. Hybrids can form between DNA-DNA, DNA-RNA or RNA-RNA. They can form between a short strand and a long strand containing a region complementary to the short one. Imperfect hybrids can also form, but the more imperfect they are, the less stable they will be (and the less likely to form).

The term “identification index” refers generally to a series of nucleotides that are incorporated into an oligonucleotide during oligonucleotide synthesis for identification purposes. Identification index sequences are preferably 6 or more nucleotides in length. In a preferred aspect, the identification index is long enough to have statistical probability of labeling each molecule with a selected genomic region uniquely. For example, if there are 3000 copies of a particular selected genomic region, there are substantially more than 3000 identification indexes such that each copy of a particular selected genomic region is likely to be labeled with a unique identification index. The identification index may contain additional nucleotides that allow for identification and correction of sequencing errors including the detection of deletion, substitution, or insertion of one or more bases during sequencing as well as nucleotide changes that may occur outside of sequencing such as oligo synthesis, amplification, and any other aspect of the assay. The index may be combined with any other index to create one index that provides information for two properties (e.g., sample-identification index, allele-locus index).

As used herein the term “ligase” refers generally to a class of enzymes, which can link pieces of nucleic acid together. “Ligation” is the process of joining pieces of DNA together.

The terms “locus” and “loci” as used herein refer to genomic regions of known location in a genome.

The term “locus index” refers generally to a series of nucleotides that correspond to a given locus. In one aspect, the locus index is long enough to label each locus interrogated by the assay systems uniquely. In another aspect, a single locus index can be used for two or more loci in a predetermined subchromosomal region. The term “maternal sample” as used herein refers to any sample taken from a pregnant mammal which comprises both fetal and maternal DNA. Preferably, maternal samples for use in the invention are obtained through relatively non-invasive means, e.g., phlebotomy or other standard techniques for extracting peripheral samples from a subject.

The term “melting temperature” or Tₘ is commonly defined as the temperature at which a population of double-stranded nucleic acid molecules becomes half dissociated into single strands. The equation for calculating the Tₘ of nucleic acids is well known in the art. As indicated by standard references, a simple estimate of the Tₘ value may be calculated by the equation: Tₘ=81.5+16.6(log 10[Na+])0.41(%[G+C])−675/n−1.0m, when a nucleic acid is in aqueous solution having cation concentrations of 0.5 M or less, the (G+C) content is between 30% and 70%, n is the number of bases, and m is the % age of base pair mismatches (see, e.g., Sambrook J et al., Molecular Cloning, A Laboratory Manual, 3rd Ed., Cold Spring Harbor Laboratory Press (2001)). Other references include more sophisticated computations, which take structural as well as sequence characteristics into account for the calculation of Tₘ.

“Microarray” or “array” refers to a solid phase support having a surface, preferably but not exclusively a planar or substantially planar surface, which carries an array of sites containing nucleic acids such that each site of the array comprises substantially identical or identical copies of oligonucleotides or polynucleotides and is spatially defined and not overlapping with other member sites of the array; that is, the sites are spatially discrete. The array or microarray can also comprise a non-planar interrogatable structure with a surface such as a bead or a well. The oligonucleotides or polynucleotides of the array may be covalently bound to the solid support, or may be non-covalently bound. Conventional microarray technology is reviewed in, e.g., Schena, Ed., Microarrays: A Practical Approach, IRL Press, Oxford (2000). “Array analysis”, “analysis by array” or “analysis by microarray” refers to analysis, such as, e.g., sequence analysis, of one or more biological molecules using a microarray.

The term “non-polymorphic”, when used with respect to detection of selected loci, is meant a detection of such locus, which may contain one or more polymorphisms, but in which the detection is not reliant on detection of the specific polymorphism within the region. Thus a selected locus may contain a polymorphism, but detection of the region using the assay system of the invention is based on occurrence of the region rather than the presence or absence of a particular polymorphism in that region.

The term “oligonucleotides” or “oligos” as used herein refers to linear oligomers of natural or modified nucleic acid monomers, including deoxyribonucleotides, ribonucleotides, anomeric forms thereof, peptide nucleic acid monomers (PNAs), locked nucleotide acid monomers (LNA), and the like, or a combination thereof, capable of specifically binding to a single-stranded polynucleotide by way of a regular pattern of monomer-to-monomer interactions, such as Watson-Crick type of base pairing, base stacking, Hoogsteen or reverse Hoogsteen types of base pairing, or the like. Usually monomers are linked by phosphodiester bonds or analogs thereof to form oligonucleotides ranging in size from a few monomeric units, e.g., 8-12, to several tens of monomeric units, e.g., 100-200 or more. Suitable nucleic acid molecules may be prepared by the phosphoramidite method described by Beaucage and Carruthers (Tetrahedron Lett., 22:1859-1862 (1981)), or by the triester method according to Matteucci, et al. (J. Am. Chem. Soc., 103:3185 (1981)), both incorporated herein by reference, or by other chemical methods such as using a commercial automated oligonucleotide synthesizer.

As used herein “nucleotide” refers to a base-sugar-phosphate combination. Nucleotides are monomeric units of a nucleic acid sequence (DNA and RNA). The term nucleotide includes ribonucleoside triphosphates ATP, UTP, CTG, GTP and deoxyribonucleoside triphosphates such as dATP, dCTP, dITP, dUTP, dGTP, dTTP, or derivatives thereof. Such derivatives include, for example, [αS]dATP, 7-deaza-dGTP and 7-deaza-dATP, and nucleotide derivatives that confer nuclease resistance on the nucleic acid molecule containing them. The term nucleotide as used herein also refers to dideoxyribonucleoside triphosphates (ddNTPs) and their derivatives. Illustrated examples of dideoxyribonucleoside triphosphates include, but are not limited to, ddATP, ddCTP, ddGTP, ddITP, and ddTTP.

According to the present invention, a “nucleotide” may be unlabeled or detectably labeled by well-known techniques. Fluorescent labels and their attachment to oligonucleotides are described in many reviews, including Haugland, Handbook of Fluorescent Probes and Research Chemicals, 9th Ed., Molecular Probes, Inc., Eugene Oreg. (2002); Keller and Manak, DNA Probes, 2nd Ed., Stockton Press, New York (1993); Eckstein, Ed., Oligonucleotides and Analogues: A Practical Approach, IRL Press, Oxford (1991); Wetmur, Critical Reviews in Biochemistry and Molecular Biology, 26:227-259 (1991); and the like. Other methodologies applicable to the invention are disclosed in the following sample of references: Fung et al., U.S. Pat. No. 4,757,141; Hobbs, Jr., et al., U.S. Pat. No. 5,151,507; Cruickshank, U.S. Pat. No. 5,091,519; Menchen et al., U.S. Pat. No. 5,188,934; Begot et al., U.S. Pat. No. 5,366,860; Lee et al., U.S. Pat. No. 5,847,162; Khanna et al., U.S. Pat. No. 4,318,846; Lee et al., U.S. Pat. No. 5,800,996; Lee et al., U.S. Pat. No. 5,066,580: Mathies et al., U.S. Pat. No. 5,688,648; and the like. Labeling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; and 2003/0017264. Detectable labels include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), CASCADE BLUE® (pyrenyloxytrisulfonic acid), OREGON GREEN® (2′,7′-difluorofluorescein), TEXAS RED™ (sulforhodamine 101 acid chloride), Cyanine and 5-(2′-aminoethyl)aminonaphthalene-1-sulfonic acid (EDANS). Specific examples of fluorescently labeled nucleotides include [R6G]dUTP, [TAMRA]dUTP, [R110]dCTP, [R6G]dCTP, [TAMRA]dCTP, [JOE]ddATP, [R6G]ddATP, [FAM]ddCTP, [R110]ddCTP, [TAMRA]ddGTP, [ROX]ddTTP, [dR6G]ddATP, [dR110]ddCTP, [dTAMRA]ddGTP, and [dROX]ddTTP available from Perkin Elmer, Foster City, Calif. FluoroLink DeoxyNucleotides, FluoroLink Cy3-dCTP, FluoroLink Cy5-dCTP, FluoroLink FluorX-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, CASCADE BLUE®-7-UTP (pyrenyloxytrisulfonic acid-7-UTP), CASCADE BLUE®-7-dUTP (pyrenyloxytrisulfonic acid-7-dUTP), fluorescein-12-UTP, fluorescein-12-dUTP, OREGON GREEN® 488-5-dUTP (2′,7′-difluorofluorescein-5-dUTP), RHODAMINE GREEN™-5-UTP ((5-{2-[4-(aminomethyl)phyenyl]-5-(pyridin-4-yl)1h-I-5UTP), RHODAMINE GREEN™-5-dUTP((5-{2-[4-(aminomethyl)phyenyl]-5-(pyridin-4-yl)1h-I-5dUTP), tetramethylrhodamine-6-UTP, tetramethylrhodamine-6-dUTP, TEXAS RED™-5-UTP (sulforhodamine 101 acid chloride-5-UTP), TEXAS RED™-5-dUTP (sulforhodamine 101 acid chloride-5-dUTP, and TEXAS RED™-12-dUTP (sulforhodamine 101 acid chloride-12-dUTP available from Molecular Probes, Eugene, Oreg.

As used herein the term “polymerase” refers to an enzyme that links individual nucleotides together into a long strand, using another strand as a template.

As used herein “polymerase chain reaction” or “PCR” refers to a technique for amplifying a specific piece of target DNA in vitro, even in the presence of excess non-specific DNA.

The term “polymorphism” as used herein refers to any genetic changes or variants in a loci that may be indicative of that particular loci, including but not limited to single nucleotide polymorphisms (SNPs), methylation differences, short tandem repeats (STRs), and the like.

Generally, a “primer” is an oligonucleotide used to, e.g., prime DNA extension, ligation and/or synthesis, such as in the synthesis step of the polymerase chain reaction or in the primer extension techniques described herein.

The term “reference chromosome” as used herein refers to a chromosome that is used for comparison to a chromosome of interest in a particular sample. In certain preferred aspects, a chromosome may be both a chromosome of interest, in that it is commonly associated with a copy number variation such as aneuploidy, and a reference chromosome for a different chromosome of interest.

The terms “sequencing” as used herein refers generally to any and all biochemical methods that may be used to determine the order of nucleotide bases including but not limited to adenine, guanine, cytosine and thymine, in one or more molecules of DNA. As used herein the term “sequence determination” means using any method of sequencing known in the art to determine the sequence nucleotide bases in a nucleic acid.

The term “value of the probability” refers to any value achieved by directly calculating probability or any value that can be correlated to or otherwise is indicative of a probability.

### DETAILED DESCRIPTION OF THE INVENTION

The practice of the techniques described herein may employ, unless otherwise indicated, conventional techniques and descriptions of organic chemistry, polymer technology, molecular biology (including recombinant techniques), cell biology, biochemistry, and sequencing technology, which are within the skill of those who practice in the art. Such conventional techniques include polymer array synthesis, hybridization and ligation of polynucleotides, and detection of hybridization using a label. Specific illustrations of suitable techniques can be had by reference to the examples herein. However, other equivalent conventional procedures can, of course, also be used. Such conventional techniques and descriptions can be found in standard laboratory manuals such as Green, et al., Eds. (1999), Genome Analysis: A Laboratory Manual Series (Vols. I-IV); Weiner, Gabriel, Stephens, Eds. (2007), Genetic Variation: A Laboratory Manual; Dieffenbach, Dveksler, Eds. (2003), PCR Primer: A Laboratory Manual; Bowtell and Sambrook (2003), DNA Microarrays: A Molecular Cloning Manual; Mount (2004), Bioinformatics: Sequence and Genome Analysis; Sambrook and Russell (2006), Condensed Protocols from Molecular Cloning: A Laboratory Manual; and Sambrook and Russell (2002), Molecular Cloning: A Laboratory Manual (all from Cold Spring Harbor Laboratory Press); Stryer, L. (1995) Biochemistry (4th Ed.) W.H. Freeman, New York N.Y.; Gait, “Oligonucleotide Synthesis: A Practical Approach” 1984, IRL Press, London; Nelson and Cox (2000), Lehninger, Principles of Biochemistry 3^{rd} Ed., W. H. Freeman Pub., New York, N.Y.; and Berg et al. (2002) Biochemistry, 5^{th} Ed., W.H. Freeman Pub., New York, N.Y., all of which are herein incorporated in their entirety by reference for all purposes.

Note that as used herein and in the appended claims, the singular forms “a,” “an,” and “the” include plural referents unless the context clearly dictates otherwise. Thus, for example, reference to “an allele” refers to one or more copies of allele with various sequence variations, and reference to “the assay system” includes reference to equivalent steps and methods known to those skilled in the art, and so forth.

Unless defined otherwise, all technical and scientific terms used herein have the same meaning as commonly understood by one of ordinary skill in the art to which this invention belongs. All publications mentioned herein are incorporated by reference for the purpose of describing and disclosing devices, formulations and methodologies that may be used in connection with the presently described invention.

Where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

In the following description, numerous specific details are set forth to provide a more thorough understanding of the present invention. However, it will be apparent to one of skill in the art that the present invention may be practiced without one or more of these specific details. In other instances, features and procedures well known to those skilled in the art have not been described in order to avoid obscuring the invention.

### The Invention in General

The invention provides assay systems and methods to identify copy number variants of selected genomic regions of interest (including loci, sets of loci and larger genomic regions, e.g., chromosomes), mutations, and polymorphisms in selected genomic regions of interest in a sample using digital PCR techniques.

In one aspect, the assay system utilizes methods to selectively identify and/or isolate two or more genomic regions of interest (e.g., chromosomes or loci) in a sample, allowing determination of an atypical copy number of a particular genomic region based on the comparison of the relative frequencies of digital PCR-detected selected genomic regions of interest from two or more chromosomes in the sample or by comparison to one or more reference chromosomes from the same or a different sample.

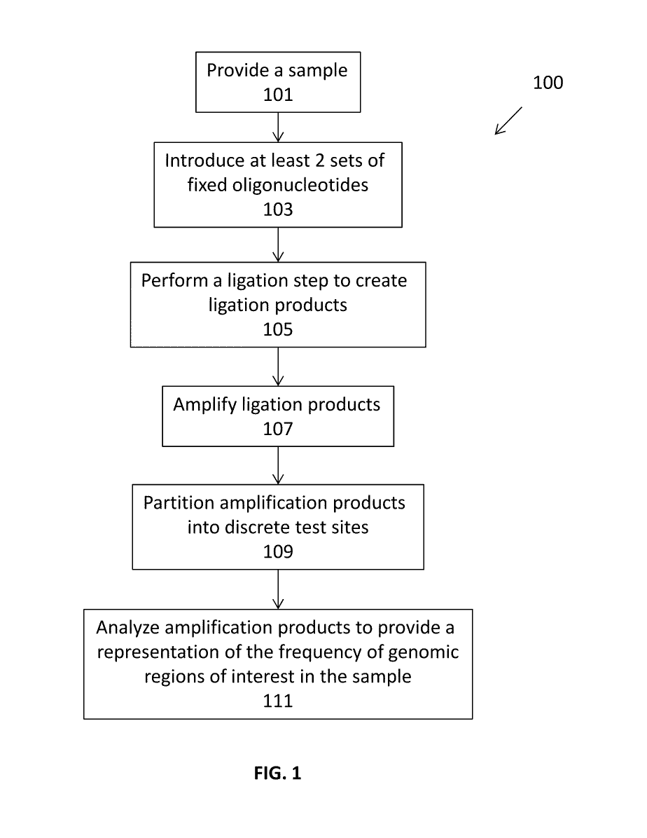

FIG. 1 is a simplified flow chart of the general steps utilized in detection and quantification of selected genomic regions of interest in a sample. FIG. 1 shows method 100, where in a first step 101, a sample is provided comprising a major source and a minor source of cell-free DNA. In certain aspects, the sample may be a maternal sample provided from a pregnant woman comprising maternal and fetal cell-free DNA. For example, the sample may be a maternal sample in the form of whole blood, plasma, or serum. In certain other aspects, the sample may be a sample from a patient that has received a non-autologous transplant. Optionally, the cell-free DNA is isolated from the sample prior to further analysis. At step 103, two or more sets of first and second fixed sequence oligonucleotides are introduced to the sample under conditions that allow the sets of fixed sequence oligonucleotides to specifically hybridize to complementary regions in the genomic regions of interest.

At step 105, a ligation step is performed to create a ligation product. At step 107, the ligation product is amplified. Amplification may be accomplished through the use of universal primer sequences. Amplification products may be detected directly from the amplification reaction, or they are optionally diluted in preparation for detection. At step 109, the amplification products are partitioned into a plurality of discrete test sites. The amplification products are partitioned such that most of the plurality of discrete test sites comprises either one or zero amplification products.

At step 111, the amplification products are analyzed to provide a representation of the frequency of the selected genomic regions of interest in the sample. Analysis of the amplification products can be carried out, for example, in each discrete test site where detection of an amplification product within the discrete test site results in a positive test indication for that discrete site.

### Detection of Non-Polymorphic Genomic Regions Using a Ligation-Based Assay System and Digital PCR Detection System

The assay system of the present invention interrogates selected genomic regions of interest. In certain general aspects, the assay system uses sets of two fixed sequence oligonucleotides fully complementary to selected genomic regions of interest on chromosomes of interest. The fixed sequence oligonucleotides in a set may hybridize adjacently to one another in a selected genomic region, or the fixed sequence oligonucleotides of a set may hybridize nonadjacently to a selected genomic region leaving a non-hybridized region between the two fixed sequence oligonucleotides. In the latter case, the region between the two fixed sequence oligonucleotides may be filled by primer extension, or by employment of a third, bridging oligonucleotide that hybridizes to the region between the fixed sequence oligonucleotides.

In certain general aspects, the assay system utilizes a ligation method comprising the use of sets of first and second fixed sequence oligonucleotides complementary to selected genomic regions on a chromosome of interest or a reference chromosome

One or more short, bridging oligonucleotides complementary to the region between and immediately adjacent to the first and second fixed sequence oligonucleotides of each set may also be used. Hybridization of the sets of fixed sequence oligonucleotides, and optionally bridging oligonucleotides, to selected genomic regions of interest followed by ligation of the oligonucleotides, provides a template for further amplification, detection and quantification of the selected genomic regions using digital PCR.

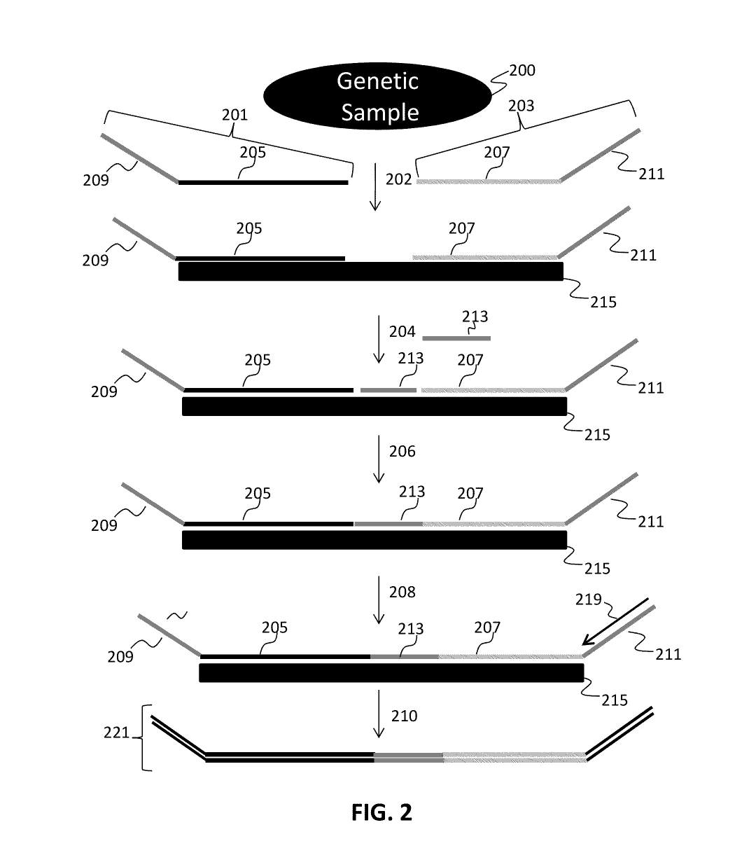

In a preferred aspect, the assay system of the invention employs a multiplexed reaction with a set of three or more oligonucleotides for each selected genomic region. This general aspect is illustrated in FIG. 2. Each set of oligonucleotides preferably contains two oligonucleotides 201, 203 of fixed sequence and one or more bridging oligonucleotides 213.

Each of the fixed sequence oligonucleotides comprises a region complementary to the selected genomic region 205, 207. At least one fixed sequence oligonucleotide 201 comprises an index region 209 which can be used in detection techniques to quantify the genomic regions of interest. At least one fixed sequence oligonucleotide comprises a universal primer sequence (here, 211), i.e. regions in the fixed sequence oligonucleotides complementary to universal primers. The at least one universal primer sequence 211 is used to amplify the different selected genomic regions following ligation of the hybridized fixed sequence oligonucleotides and the bridging oligonucleotide. The at least one universal primer region is located at or near the end of fixed sequence oligonucleotide 203, and thus preserves the nucleic acid-specific sequences in the products of any universal amplification methods. Though in this exemplary embodiment the universal primer 211 is shown only on fixed sequence oligonucleotide 203, universal primers may be disposed on only fixed sequence oligonucleotide 201, or on both fixed sequence oligonucleotides. Amplification products can be detected by methods such as digital PCR, determination of the sequence of the products, e.g., through next generation sequence determination or by hybridization, e.g., to an array or a bead-based detection system such as the Luminex™ bead-based assay (Invitrogen, Carlsbad, Calif.) or the BeadXpress™ assay (Illumina, San Diego, Calif.).

In one aspect of the assay systems of the invention, the fixed sequence oligonucleotides 201, 203 are introduced 202 to the sample 200 and allowed to specifically bind to complementary genomic regions of interest 215 in the sample. Following hybridization, unhybridized fixed sequence oligonucleotides are preferably separated from the remainder of the sample (not shown). The bridging oligonucleotides are then introduced and allowed to bind 204 to the region of the selected genomic region 215 between the first 201 and second 203 fixed sequence oligonucleotides. Alternatively, the bridging oligo can be introduced simultaneously with the fixed sequence oligonucleotides. The bound oligonucleotides are ligated 206 to create a ligation product spanning and complementary to the genomic region of interest. Following ligation, at least one universal primer 219 is introduced to amplify 208 the ligation product to create 210 amplification products 221 that comprise the sequence of the genomic region of interest. These products 221 are optionally isolated, detected, and quantified to provide frequency information of the selected genomic regions of interest in a sample. Preferably, the products are detected and quantified through digital PCR.

In certain aspects, the assay system utilizes a tandem ligation method comprising the use of at least two sets of first and second fixed sequence oligonucleotides complementary to selected genomic regions on both a first and a second chromosome. One or more short, bridging oligonucleotides complementary to the region between and immediately adjacent to the first and second fixed sequence oligonucleotides of each set may also be used. Hybridization of these sets of oligonucleotides to the selected genomic regions of interest, followed by ligation of the oligonucleotides of each set, provides a template for amplification, detection and quantification of the selected genomic region of interest using digital PCR. The selected genomic regions may be quantified directly from the amplification products of the amplification reactions, or the amplification products are optionally isolated and identified to quantify the number of selected genomic regions from the sample.

In certain specific aspects, the assay system employs sets of first and second fixed sequence oligonucleotides that hybridize adjacent to one another with no need for an extension step or bridging oligonucleotides. The fixed sequence oligonucleotides of each set are ligated to each other during the ligation reaction resulting in a single template for further amplification and sequence determination or other read out.

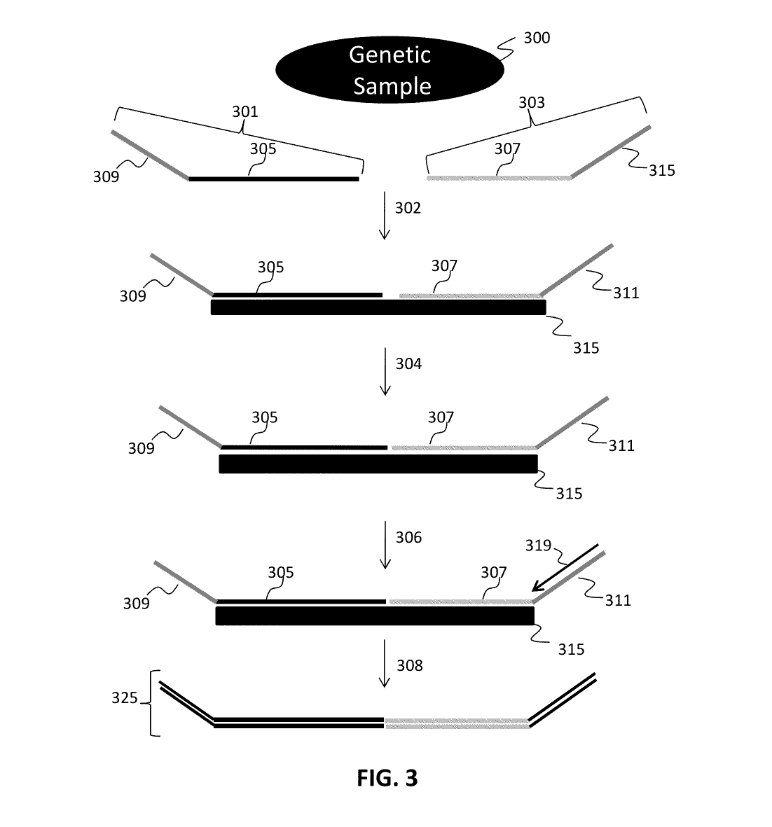

FIG. 3 illustrates an assay system of the invention which employs a set of two fixed sequence oligonucleotides 301, 302 for each selected genomic region. Each of the fixed sequence oligonucleotides comprises a region complementary to the selected nucleic acid regions 305, 307. At least one fixed sequence oligonucleotide (here, 301) comprises an index region 309 which can be used in detection techniques to identify and quantify the selected genomic regions of interest. At least one fixed sequence oligonucleotide (here, 303 comprises a universal primer sequence 311, i.e. an oligonucleotide region complementary to universal primers. The at least one universal primer region is used in a later step to amplify the ligated fixed sequence oligonucleotides of each step.

In one aspect of the assay systems of the invention, the fixed sequence oligonucleotides 301, 303 are introduced 302 to the sample 300 and allowed to specifically bind to complementary genomic regions of interest 315 adjacent to one another. Following hybridization, unhybridized fixed sequence oligonucleotides are preferably separated from the remainder of the sample (not shown). The hybridized fixed sequence oligonucleotides of each set are ligated 304 to create a ligation product spanning and complementary to the genomic regions of interest. Following ligation, at least one universal primer 319 is introduced to amplify 306 the ligated fixed sequence oligonucleotides of each step to create 308 amplification products 325 that comprise the sequences of the genomic regions of interest. These amplification products 325 are optionally isolated, detected, and quantified to provide information on the presence and amount of each selected genomic region of interest in the sample.

In other certain specific aspects, the assay system utilizes first and second fixed sequence oligonucleotides that are complimentary to non-adjacent regions in the selected genomic regions of interest. Hybridization of these sets of two fixed sequence oligonucleotides to selected genomic regions of interest may be followed by an extension reaction using dNTPs and a polymerase to create a set of adjacently hybridized fixed sequence oligonucleotides. The extension reaction followed by a ligation reaction provides a template for further amplification, detection and quantification of the selected genomic regions.

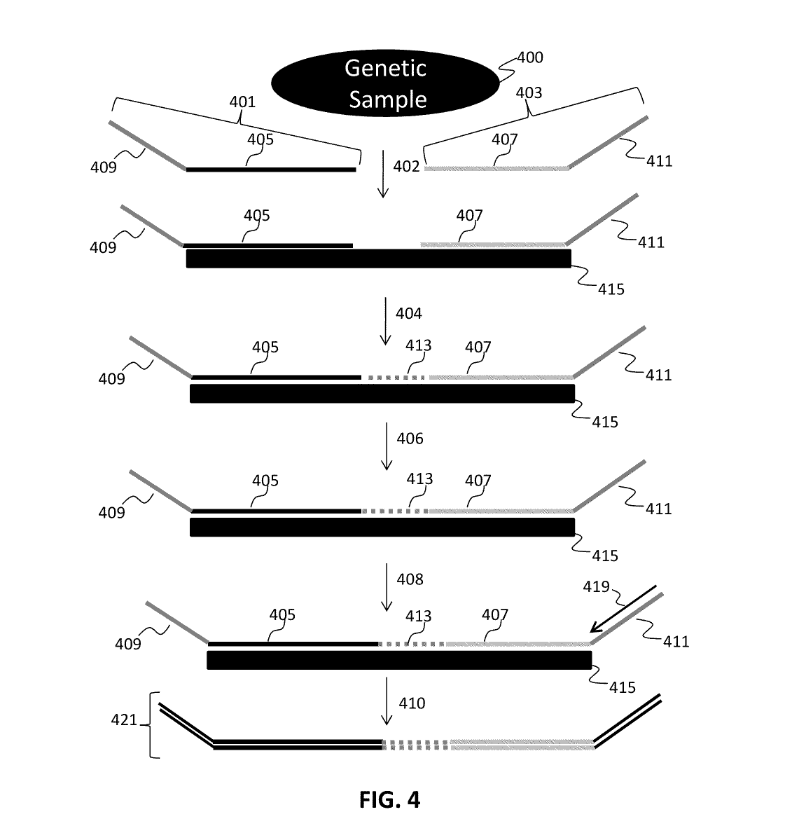

FIG. 4 illustrates an assay system of the invention which employs a set of two fixed sequence oligonucleotides 401, 403 for each selected genomic region. Each of the fixed sequence oligonucleotides comprises a region complementary to the selected genomic region 405, 407. At least one fixed sequence oligonucleotide (here, 401) comprises an index region 409 which can be used in detection techniques to quantify genomic regions of interest. At least one fixed sequence oligonucleotide (here, 403) comprises a universal primer sequence 411, i.e., and oligonucleotide region common to many if not all of one fixed sequence oligonucleotide of each set and complementary to a universal primer. The universal primer sequence is used to amplify the selected genomic regions of interest following ligation of the hybridized fixed sequence oligonucleotides.

In one aspect of the assay systems of the invention, the fixed sequence oligonucleotides 401, 403 are introduced 402 to the sample 400 and allowed to specifically hybridize to complementary genomic regions of interest 415 such that the fixed sequence oligonucleotides 401, 403 are not hybridized adjacent to one another. Following hybridization, unhybridized fixed sequence oligonucleotides are preferably separated from the sample (not shown). An extension reaction 404 is carried out using dNTPs and a polymerase to create a set of adjacently hybridized oligonucleotides. The adjacently hybridized oligonucleotides are ligated 406 to create ligation products spanning and complementary to the genomic regions of interest. Following ligation, at least one universal primer 419 is introduced to amplify 408 the ligated oligonucleotides to create 410 amplification products 425 that comprise the sequence of the genomic region of interest. These amplification products 425 are optionally isolated, detected, and quantified to provide information on the presence and amount of the selected genomic regions in a sample.

In other certain specific aspects, the assay system utilizes a tandem ligation method comprising the use of sets of first and second fixed sequence oligonucleotides complementary to selected genomic regions on a chromosome of interest or a reference chromosome and one or more short, bridging oligonucleotides complementary to the region between and immediately adjacent to the region in the selected genomic regions of interest complementary to the first and second fixed sequence oligonucleotides. Hybridization of these sets of three or more oligonucleotides to selected genomic regions of interest followed by ligation provides a ligation product suitable for amplification, detection and quantification using digital PCR. The amplified regions may be quantified directly from the amplification reactions, or they are optionally isolated and identified to quantify the number of selected genomic regions in a sample.

In specific aspects, the tandem ligation methods use sets of fixed sequence oligonucleotides with a set of two or more bridging oligonucleotides that hybridize contiguously and adjacently to the selected genomic region between the regions complementary to the fixed sequence oligonucleotides. In this embodiment, the bridging oligonucleotides hybridize adjacent to one another and to the fixed sequence oligonucleotides. The bridging oligonucleotides are ligated during the ligation reaction with the fixed sequence oligonucleotides and with each other, resulting in a single ligation product for each selected genomic region for further amplification and sequence determination.

In other aspects of the invention, the assay system uses a set of fixed sequence oligonucleotides that bind to non-adjacent regions within a genomic region of interest, and primer extension is utilized to create a contiguous set of hybridized oligonucleotides prior to the tandem ligation step. In such aspects, the assay system utilizes a tandem ligation method comprising the use of first and second fixed sequence oligonucleotides that hybridize non-adjacently to a selected genomic region on a chromosome of interest or a reference chromosome, and one or more short, bridging oligonucleotides that hybridize to a region between the first and second fixed sequence oligonucleotides but not immediately adjacent to either of the fixed sequence oligonucleotides. Hybridization of these sets of three or more oligonucleotides (fixed and bridging) to a selected genomic region of interest is followed by an extension reaction using dNTPs and a polymerase to create a set of adjacently hybridized oligonucleotides, and the adjacently hybridized oligonucleotides are then ligated to one another. The combination of extension and ligation provides a ligation product that can be used as a template for amplification, detection and quantification of the selected genomic regions. The amplified selected genomic regions may be quantified directly from the amplification reactions, or the amplified selected genomic regions are optionally isolated and identified to quantify the number of selected genomic regions in a sample.

In specific aspects, the tandem ligation methods use sets of two fixed sequence oligonucleotides with a set of two or more sequential bridging oligonucleotides that hybridize non-adjacently to the region of the nucleic acid between the region complementary to the fixed sequence oligonucleotides. The “gap” regions between the fixed sequence oligonucleotides and the bridging oligos and/or between the sequential bridging oligonucleotides are ligated during the ligation reaction, resulting in a single ligation product which can be used as a template for amplification and sequence determination.

In preferred aspects of the invention, the nucleic acids from the sample are associated with a substrate, e.g., using binding pairs to attach or immobilize the genetic material to a substrate surface. Briefly, a first member of a binding pair (e.g., biotin) can be associated with a nucleic acid of interest, and the associated nucleic acid attached to a substrate comprising a second member of a binding pair (e.g., avidin or streptavidin) on its surface. Immobilization can be particularly useful in removing any unhybridized oligonucleotides following hybridization of the sets of fixed sequence oligonucleotides and/or the bridging oligonucleotides to the genomic region of interest. Briefly, the immobilized nucleic acids can be hybridized to the sets of oligonucleotides, and treated or processed to remove any unhybridized oligonucleotides, e.g., by washing or other removal methods such as degradation of unhybridized oligonucleotides as discussed in Willis et al., U.S. Pat. Nos. 7,700,323 and 6,858,412.

There are a number of methods that may be used in the immobilization of a nucleic acid via binding pair interactions, as will be apparent to one skilled in the art upon reading the present specification. For example, numerous methods may be used for labeling the nucleic acids of a sample with biotin, including random photobiotinylation, end-labeling with biotin, replicating with biotinylated nucleotides, and replicating with a biotin-labeled primer.

The number of selected genomic regions analyzed for each chromosome in the assay system of the invention may vary from 2-20,000 or more per chromosome analyzed. In a preferred aspect, the number of selected genomic regions is between 48 and 480. In another aspect, the number of selected genomic regions is at least 100. In another aspect, the number of selected genomic regions is at least 400. In another aspect, the number of selected genomic regions is at least 1000.

In certain aspects, the bridging oligonucleotides can be composed of mixture of oligonucleotides with degeneracy in each of the positions, so that the mixture of random sequence bridging oligonucleotides used will be compatible with all reactions in the multiplexed assay requiring bridging oligonucleotides of a given length. In another aspect, the bridging oligonucleotides can be of various lengths so that the mixture of random sequence bridging oligonucleotides will be compatible with particular tandem ligation reactions in the multiplexed assay requiring bridging oligos of different lengths.

In yet another aspect the bridging oligonucleotide can have partial degeneracy and the multiplexed tandem ligation reactions are restricted to those that require the specific sequences provided by the degeneracy of the bridging oligonucleotides. For example, a set of tandem ligation reactions may require only A and C bases in the bridging oligonucleotide, and a mixture of bridging oligonucleotides synthesized with only A and C bases would be provided for these particular tandem ligation reactions in a multiplexed assay.

In yet another aspect, the bridging oligonucleotide sequences are designed such that only those assays that have given, specific sequences in the bridging region would be multiplexed in the assay system. In one example the bridging oligonucleotide is a randomer, where all combinations of the bridging oligonucleotide are synthesized. As an example, in the case where a 5-base bridging oligonucleotide is used, the number of unique bridging oligonucleotides would be 4̂5=1024. This would be independent of the number of selected genomic regions since all possible bridging oligonucleotides would be present in the reaction.

In another example the bridging oligonucleotides are specific, synthesized to match the sequences in the gap between fixed sequence oligonucleotides of each set. As an example, in the case where a 5-base bridging oligonucleotide is used, the number of unique oligonucleotides synthesized would be equal to or less than the number of selected genomic regions. A number less than the number of selected genomic regions could be achieved if the gap sequence was shared between two or more selected genomic regions. In one aspect of this example, one might purposefully choose the selected genomic region sequences and particularly the gap sequences such that there was as much overlap as possible in the gap sequences, minimizing the number of bridging oligonucleotides necessary for the multiplexed reaction.

In another aspect, the sequences of the bridging oligonucleotides are designed and the selected genomic regions are selected so that all selected genomic regions share the same base(s) at each end of the bridging oligonucleotide. For instance, one might choose selected genomic regions with a gap location such that all of the gaps share an “A” base at the first position and a “G” base at the last position of the gap. Any combination of a first and last base could be utilized, based upon factors such as the genome investigated, the likelihood of sequence variation in that area, and the like. In a specific aspect of this example, the bridging oligonucleotides can be synthesized by random degeneracy of bases at the internal positions of the bridging oligonucleotide, specific addition at the first and last position. In the case of a 5-mer, the second, third and fourth positions would be randomly provided, and two specific nucleotides would be added at the proximal positions. In this case, the number of unique bridging oligonucleotides would be 4̂3=64.

In the human genome, the frequency of the dinucleotide CG is much lower than expected by the respective mononucleotide frequencies. This presents an opportunity to enhance the specificity of an assay with a particular mixture of bridging oligonucleotides. In this aspect, the bridging oligonucleotides may be selected to have a 5′ G and a 3′ C. This base selection allows each bridging oligonucleotide to have a high frequency in the human genome but makes it a rare event for two bridging oligonucleotides to hybridize adjacent to each other. The probability is then reduced that multiple oligonucleotides are ligated in locations of the genome that are not targeted in the assay.

The bridging oligonucleotides are preferably added to the reaction after the sets of fixed sequence oligonucleotides have been hybridized, and following the optional removal of all unhybridized fixed sequence oligonucleotides. The conditions of the hybridization reaction are preferably optimized near the Tₘ of the bridging oligonucleotide to prevent erroneous hybridization of bridging oligonucleotides that are not fully complementary to the genomic region. If the bridging oligonucleotides have a Tₘ significantly lower than the fixed sequence oligonucleotides, the bridging oligonucleotide is preferably added as a part of the ligase reaction.

The advantage of using short bridging oligonucleotides is that ligation on either end would likely occur only when all bases of the bridging oligonucleotide match the gap sequence. A further advantage of short bridging oligonucleotides is that the number of different bridging oligonucleotides necessary could be less than the number of targeted selected genomic regions, raising the bridging oligonucleotides effective concentration to allow perfect matches to happen faster. Use of fewer bridging oligonucleotides also has advantages in cost and quality control. The advantages of using bridging oligonucleotides with fixed first and last bases and random bases in between include the ability to utilize longer bridging oligonucleotides for greater specificity while reducing the number of total bridging oligonucleotides required for the assay.

### Digital PCR

Detection and quantification of genomic regions of interest is preferably carried out by digital PCR. In general aspects, digital PCR is carried out by partitioning a dilute sample into a plurality of discrete test sites such that most of the plurality of discrete test sites comprises one or zero nucleic acid sequences such as amplification products. Amplification products are then analyzed and quantified, resulting in a representation of the presence or absence of genomic regions of interest corresponding to a chromosome of interest or a reference chromosome. The number of nucleic acid sequences corresponding to a chromosome of interest or a reference chromosome can then be quantified to estimate the frequency of the selected genomic regions of interest in a sample corresponding to each chromosome. Information regarding the relative frequency of genomic regions of interest can be used to determine the presence or absence of copy number variations, polymorphisms and mutations.

In certain aspects, as described above, amplification products are diluted and partitioned into a plurality of discrete test sites. In certain other embodiments, the samples are not diluted before the sample is partitioned into a plurality of discrete test sites. In certain embodiments, the amplification products are partitioned such that on average, there is a distribution of less than one amplification product per test well. In such embodiments, analysis of each discrete testing site provides an indication of the presence or absence of an amplification product.

Discrete test sites may comprise any suitable form for a particular application. Examples of carriers for suitable discrete test sites include, but are not limited to, micro well plates, dispersed phase of an emulsion, arrays of miniaturized chambers, capillaries, and nucleic acid binding surfaces.

The number of discrete test sites and the number of samples may vary depending on the application and the level of statistical confidence to be achieved. The number of discrete test sites employed in the analysis may also depend on the level of minor-source DNA in the sample. In certain aspects, the number of discrete test sites employed may be between 200 and 20 million, such as 20,000 and 20 million, or even more specifically, between 200,000 and 20 million. In certain specific embodiments, the number of discrete test sites may be more than 20 million. The volume capability of the discrete test sites may vary depending on the application. In certain embodiments, a discrete test site can hold a volume of 1-100 μL.

In some aspects, analysis of the amplification products comprises analysis of an index such as a chromosomal index that is provided on the amplification product. In certain aspects, fluorescence techniques are used to distinguish the presence or absence of certain nucleic acid sequences in discrete test sites.

In some aspects, different amplification products from the same chromosome have the same chromosomal index. In certain embodiments, 10 amplification products from the same chromosome have the same chromosomal index. In certain embodiments, 100 amplification products from the same chromosome have the same chromosomal index. In certain embodiments, 200 amplification products from the same chromosome have the same chromosomal index. In certain embodiments, 500 amplification products from the same chromosome have the same chromosomal index. In certain embodiments, more than 500 amplification products from the same chromosome have the same chromosomal index.

As described above, the sample is partitioned such that, on average, there is a distribution of less than one amplification product per test well. Most discrete test sites comprise either one amplification product or zero amplification products. In certain embodiments, each discrete test site has the possibility of having zero amplification products, one amplification product, or more than one amplification product. In certain embodiments, if more than one amplification product is detected in a discrete test site, the information for that site is considered non-informative and is not used in further data analysis. Additionally, in certain aspects, if zero targets are detected in a discrete test site, the information for that site is considered non-informative and is not used in further data analysis. Upon detection, the discrete test sites will provide a binary “yes-or-no” result indicating the presence or absence of a particular nucleic acid sequence.

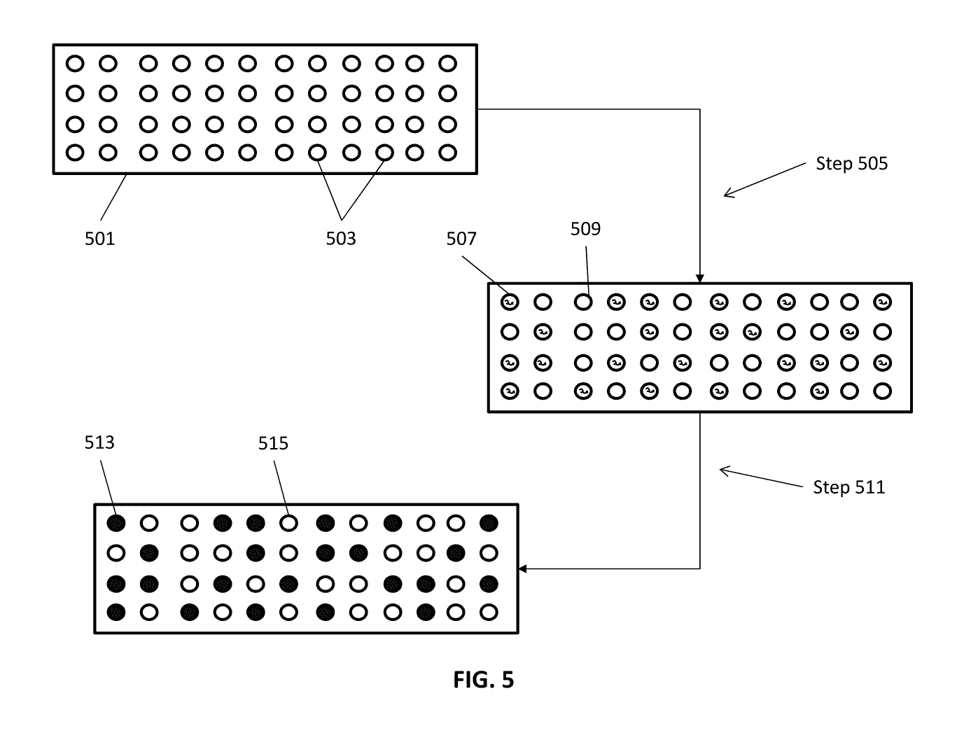

FIG. 5 illustrates simplified general steps in detection of amplification products using digital PCR. Digital PCR employs a plurality of discrete test sites 503 on a test site carrier 501. During step 505, amplification products are partitioned into the discrete test sites 503. The amplification products are partitioned such that most discrete test sites 503 comprise either one amplification product 507 or zero amplification products 509. When detection occurs at step 511, the digital PCR results indicate the presence 513 or absence 515 of a reaction.

Data reflecting the presence or absence of amplification reactions can be used to determine relative frequencies of genomic regions of interest in the original sample and/or relative frequencies of chromosomes in the sample.

In certain aspects, digital PCR is used to directly detect genomic regions of interest. In certain aspects, however, the selected genomic regions of interest are associated with one or more indices that are identifying for the selected genomic regions. The detection of the one or more indices can serve as a surrogate detection mechanism for the selected genomic region or as confirmation of the presence of a genomic region, such as a genomic region from a particular chromosome. In certain embodiments, both the index and the genomic region itself are detected. Indices are preferably associated with the selected genomic regions during the ligation step using oligonucleotides (usually one of the fixed sequence oligonucleotides) that comprise both the index and the sequence-specific regions that hybridize to the selected genomic regions.

In one example, one or both of the fixed sequence oligonucleotides used for hybridization to the selected genomic regions are designed to provide a chromosomal index. In certain aspects, the chromosomal index is unique for a chromosome of interest or a reference chromosome and is associated with each of the selected genomic regions of interest corresponding to that chromosome, so that quantification of the chromosomal index in a sample provides quantification data for the selected genomic regions on that chromosome.

In certain aspects, only the chromosomal index is detected and used to quantify the selected genomic regions in a sample. In certain aspects, a count of the number of times each chromosomal index occurs is carried out to determine the relative frequency of each chromosome in a sample.

In certain aspects, one or both of the fixed sequence oligonucleotides used for hybridization to the selected genomic regions are designed to provide a locus index. The locus index may be unique for each selected genomic region and representative of the locus on a chromosome of interest and/or a reference chromosome, so that quantification of the locus index in a sample provides quantification data for the specific locus and the particular chromosome containing the specific locus. Alternatively, the locus index can be indicative of a predetermined subchromosomal region, and thus multiple genomic regions contained within the predetermined subchromosomal regions may be identified using a single locus index.

In addition to chromosomal indices and locus-specific indices, additional indices can be used in the methods of the invention. These additional indices may be included in the one or more fixed sequence oligonucleotides, or may be introduced into an amplification product via universal primers. For example, sample indices may be used to allow for the multiplexing of samples. In addition, indices that identify sequencing errors that allow for highly multiplexed identification techniques or that allow for hybridization, ligation or attachment to a surface of, e.g., an array can be included in the amplification products. The order and placement of the indices, as well as the length of these indices can vary.

The indices used for identification and quantification of the selected genomic regions may be associated with one or both of the fixed sequence oligonucleotides used to amplify the ligation products.

The primer regions and indices in the fixed sequence oligonucleotides are preferably placed so that the indices comprising identifying information are coded at the ends of the fixed sequence oligonucleotides flanking the region complementary to the genomic regions of interest. The indices are non-complementary and unique sequences used within the one or both fixed sequence oligonucleotides to provide information relevant to the selected genomic region that is isolated and amplified using the fixed sequence oligonucleotides. The advantage is that information on the presence and quantity of the selected genomic region can be obtained without the need to detect the actual sequence itself, although in certain aspects it may be desirable to do so.

The ability to identify chromosomal frequency by using a single chromosomal index for multiple genomic regions on a chromosome reduces the sampling and assay noise and/or bias that may be associated with a specific genomic region through the use of statistical averaging. This is particularly important when using a digital PCR detection mechanism as individual multiplexing with digital PCR in its current state may be limited to performing 10 or less reactions per run which may limit the statistical strength of the results. Another advantage of the use of chromosomal indices in digital PCR detection mechanisms is that the digital PCR reaction may be optimized separately from the interrogation of the genomic regions. Digital PCR reactions can be more difficult to optimize than the interrogation system because the former involves an exponential reaction and the latter a linear replication.

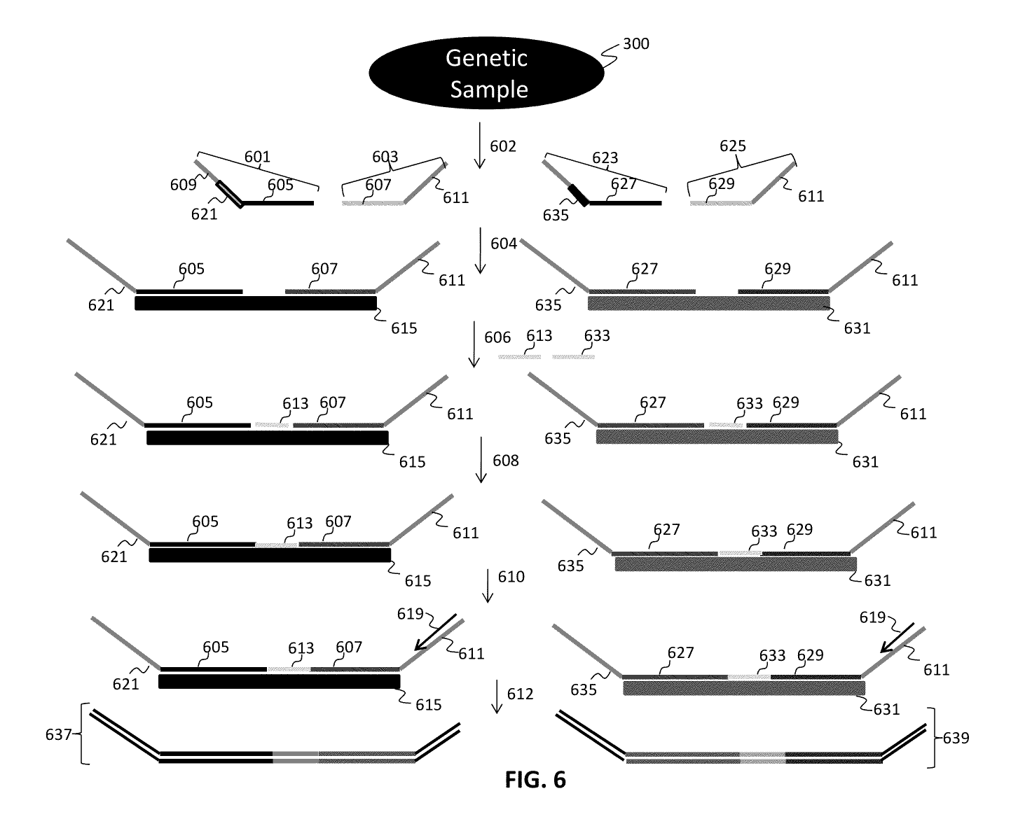

FIG. 6 illustrates the use of indices where genomic regions from two separate chromosomes are being simultaneously detected in a single tandem ligation reaction assay. Two sets of fixed sequence oligonucleotides (601 and 603, 623 and 625) that specifically hybridize to two different selected genomic regions 615, 631 are introduced 602 to a sample and allowed to hybridize 604 to the respective selected genomic regions. Each set comprises an oligonucleotide 601, 623 having a sequence specific region 605, 627, and a chromosomal index 621, 635. The other fixed sequence oligonucleotide in each set comprises a sequence specific region 607, 629 and a universal primer region 611. Following hybridization, the unhybridized fixed sequence oligonucleotides are preferably separated from the remainder of the sample (not shown). Bridging oligonucleotides 613, 633 are introduced to the hybridized fixed sequence oligonucleotide/genomic regions and allowed to hybridize 606 to these regions. Although shown in FIG. 6 as two different bridging oligonucleotides, in fact the same bridging oligonucleotide may be suitable for both hybridization events, or they may be two oligonucleotides from a pool of degenerate oligos that are used with multiple tandem ligation events. The hybridized oligonucleotides are ligated 608 to create a ligation product spanning and complementary to the genomic regions of interest. Following ligation, a universal primer 619 is introduced to amplify 610 the ligation products to create 612 amplification products 637, 639 that comprise the sequence of the genomic regions of interest. These amplification products 637, 639 are optionally isolated, detected and/or quantified to provide information on the presence and/or quantity of the selected genomic regions of particular chromosomes in a sample.

Like the example shown in FIG. 6, different chromosomal indices 621, 635 may be used in tandem ligation reactions to facilitate detection of genomic regions of interest on a particular chromosome. Detection of the two different chromosomal indices may be carried out by introduction of two digital PCR primers complementary to the chromosomal indices during an analysis and/or detection step. In certain aspects, digital PCR primers may be added to the amplification products before the amplification products are partitioned into discrete test sites. A PCR primer corresponding to a first chromosomal index may be labeled with a first fluorescent label and a PCR primer corresponding to a second chromosomal index may be labeled with a second fluorescent label.

The number of discrete test sites that are positive for the first type of fluorescent label may be counted and the number of discrete test sites that are positive for the second type of fluorescent label may be counted. Comparison of the detected genomic regions of interest corresponding to each chromosome provides information regarding the relative frequency of each chromosome in the sample. This information may be used to determine the probability of the presence or absence of a copy number variation.

In certain embodiments, relative frequencies of genomic regions of interest from two different chromosomes are compared. In certain specific embodiments, both chromosomes may be chromosomes of interest, with one chromosome acting effectively as a reference chromosome since it is unlikely that both chromosomes will exhibit aneuploidy in the same sample. For example, chromosomes 21 and 18 may be analyzed in a single sample. In some cases more than two chromosomes are used, and the combined potential non-aneuploid chromosomes used as a reference in comparison to the potentially aneuploid chromosome.

The indices may also be used to detect any amplification bias that occurs downstream of the initial isolation of the selected genomic regions from a sample. For instance, bias and variability can be introduced during DNA amplification, such as that seen during linear replication, universal amplification or during digital PCR detection. During linear replication, amplification or PCR detection, loci potentially will amplify at different rates or efficiencies. This may be due to the variety of primers in the amplification reaction with some having better efficiency than others in specific experimental conditions, e.g., due to the base composition, buffer conditions, or other conditions.

To correct for bias in digital PCR analysis results, analysis may be performed on genomic regions of interest from predetermined subchromosomal regions from the same chromosome in separate analysis and/or detection reactions. The results of the analyses of each predetermined subchromosomal region of the chromosome may be compared to account for bias in the assay and/or detection system. For example, in a certain aspect, genomic regions of interest from a first predetermined subchromosomal region of a first chromosome and a first predetermined subchromosomal region from a second chromosome may be analyzed in a first detection reaction. Subsequently, or in parallel, genomic regions of interest from a second predetermined subchromosomal region of the first chromosome a second predetermined subchromosomal region from the second chromosome may be analyzed in a second detection reaction. Comparison of the detection results from the first reaction and the second reaction may provide information regarding bias in a particular analysis reaction.

In certain aspects, a single analysis reaction is performed. In certain preferred aspects, more than one analysis reaction is performed and the results of the reactions are compared to determine a relative frequency of genomic regions of interest on a first and second chromosome. For example, more than two analysis reactions, such as three, four, five, six or seven reactions are performed. In certain aspects, one to one-thousand analysis reactions may be performed.

In certain embodiments, each ligation or amplification product corresponding to genomic regions of interest for a particular chromosome comprises the same chromosomal index for a single analysis reaction. For example, in a first detection reaction, the ligation or amplification products corresponding to genomic regions of interest for a first chromosome may comprise a first chromosomal index while the ligation or amplification products corresponding to genomic regions of interest for a second chromosome may comprise a second chromosomal index. To further reduce bias in the detection system, the chromosomal indices and/or the fluorescent probes used may be switched for a second reaction. For example, in a second detection reaction, the ligation or amplification products corresponding to genomic regions of interest for a first chromosome may comprise the same chromosomal index as the second chromosome of the first reaction while the ligation or amplification products corresponding to genomic regions of interest for the second chromosome may comprise the same chromosomal index as the first chromosome in the first reaction. Chromosomal indices may be alternated as described above in additional detection reactions or may be randomly assigned in additional detection reactions to reduce overall bias of the detection reactions. In certain aspects, different chromosomal indices may be used in each detection reaction for a particular sample.

Digital PCR techniques are described in U.S. Pat. No. 7,888,017 (Quake et al.) and U.S. Prov. Pat. App. 60/951,438 (Lo et al.), both of which are incorporated herein by reference in their entireties.

Other types of digital PCR are also suitable for these types of analysis. For example, bead emulsion PCR may be used, in which a beads comprising clonally amplified DNA are used in combination with primers directed at two specific chromosomes A and B. Emulsion PCR is carried out, resulting in beads comprising digital amplicons from only chromosomes A and B and thus it is only necessary to count beads that are positive for each type of chromosome. These methods are described in greater detail in Dressman et al., Proc. Natl. Acad. Sci. USA, 100, 8817 (Jul. 22, 2003) and WO 2005/010145 which are incorporated herein by reference in their entireties.

Another digital PCR method that would be suitable for these types of applications is microfluidic dilution with PCR in which samples are diluted as described above, and PCR reagents, primers, dNTPs, etc. are introduced to the diluted sample which is flowed through a plurality of channels. These channels may be separated into multiple reaction samples which are subjected to PCR thermal cycling. Quantitative detection is then carried out by detection of fluorescence. These techniques are described in greater detail in U.S. Pat. No. 6,960,437 (Enzelberger, et al.) which is incorporated herein by reference in its entirety.

### Amplification

In certain aspects of the invention, universal amplification is used to amplify the ligation products created through hybridization and ligation of the sets of fixed sequence oligonucleotides and bridging oligonucleotides, if present. Universal primer sequences are present in the ligation products so that the ligation products may be amplified in a single universal amplification reaction. These universal primer sequences are preferably introduced in the fixed sequence oligonucleotides, although they may also be added to the ends of the ligation products by a ligation reaction. The universal primer regions may be disposed on one of the two fixed sequence oligonucleotides in a set or on both of the fixed sequence oligonucleotides in a set.

The products of the entire ligation reaction or an aliquot of the ligation reaction may be used for the universal amplification. Using an aliquot allows different amplification reactions to be undertaken using the same or different conditions (e.g., polymerase, buffers, and the like), e.g., to ensure that bias is not inadvertently introduced due to experimental conditions. In addition, variations in primer concentrations may be used to effectively limit the number of sequence specific amplification cycles.

In certain aspects, the universal primer regions used in the assay system are designed to be compatible with conventional multiplexed assay methods that utilize general priming mechanisms to analyze large numbers of nucleic acids simultaneously. Such “universal” priming methods allow for efficient, high volume analysis of the quantity of genomic regions present in a sample, and allow for comprehensive quantification of the presence of genomic regions within such a sample for the determination of aneuploidy.

Examples of multiplexing methods used to amplify and/or genotype a variety of samples simultaneously include those described in Oliphant et al., U.S. Pat. No. 7,582,420.

Some aspects utilize coupled reactions for multiplex detection of nucleic acid sequences where oligonucleotides from an early phase of each process contain sequences which may be used by oligonucleotides from a later phase of the process. Exemplary processes for amplifying and/or detecting nucleic acids in samples can be used, alone or in combination, including but not limited to the methods described below, each of which are incorporated by reference in their entirety.

In certain aspects, the assay system of the invention utilizes one of the following combined selective and universal amplification techniques: (1) LDR coupled to PCR; (2) primary PCR coupled to secondary PCR coupled to LDR; and (3) primary PCR coupled to secondary PCR. Each of these aspects of the invention has particular applicability in detecting certain nucleic acid characteristics. However, each requires the use of coupled reactions for multiplex detection of nucleic acid sequence differences where oligonucleotides from an early phase of each process contain sequences which may be used by oligonucleotides from a later phase of the process.

Barany et al., U.S. Pat. Nos. 6,852,487, 6,797,470, 6,576,453, 6,534,293, 6,506,594, 6,312,892, 6,268,148, 6,054,564, 6,027,889, 5,830,711, 5,494,810, describe the use of the ligase chain reaction (LCR) assay for the detection of specific sequences of nucleotides in a variety of nucleic acid samples.

Barany et al., U.S. Pat. Nos. 7,807,431, 7,455,965, 7,429,453, 7,364,858, 7,358,048, 7,332,285, 7,320,865, 7,312,039, 7,244,831, 7,198,894, 7,166,434, 7,097,980, 7,083,917, 7,014,994, 6,949,370, 6,852,487, 6,797,470, 6,576,453, 6,534,293, 6,506,594, 6,312,892, and 6,268,148 describe the use of the ligase detection reaction with detection reaction (“LDR”) coupled with polymerase chain reaction (“PCR”) for nucleic acid detection.

Barany et al., U.S. Pat. Nos. 7,556,924 and 6,858,412, describe the use of padlock probes (also called “precircle probes” or “multi-inversion probes”) with coupled ligase detection reaction (“LDR”) and polymerase chain reaction (“PCR”) for nucleic acid detection.

Barany et al., U.S. Pat. Nos. 7,807,431, 7,709,201, and 7,198,814 describe the use of combined endonuclease cleavage and ligation reactions for the detection of nucleic acid sequences.

Willis et al., U.S. Pat. Nos. 7,700,323 and 6,858,412, describe the use of precircle probes in multiplexed nucleic acid amplification, detection and genotyping.

Ronaghi et al., U.S. Pat. No. 7,622,281 describes amplification techniques for labeling and amplifying a nucleic acid using an adapter comprising a unique primer and a barcode.

Detection of Polymorphic Regions using the Ligation-based Assay System and digital PCR Detection System

In certain aspects, the assay system of the invention detects one or more regions that comprise a polymorphism. This methodology is not primarily designed to identify a particular allele, e.g., as maternal versus fetal, but rather to ensure that different alleles corresponding to a genomic region of interest are included in the quantification methods of the invention. In certain aspects, however, it may be desirable to both use the information to count all such genomic regions or their corresponding chromosomal indices in the sample as well as to use the information on specific polymorphisms, e.g., to calculate the percent fetal DNA contained within a maternal sample, or identify the percent alleles with a particular mutation in a sample from a cancer patient. Information on the percent of minor source DNA in a sample may be beneficial as it provides important information on the expected statistical presence of genomic regions and variation from that expectation may be indicative of copy number variation. This may be especially helpful in circumstances where the level of minor-source DNA in a sample is low, as the percent contribution of the minor source can be used to determine quantitative statistical significance in the variations of levels of identified genomic regions in the sample. In other aspects, determination of the percent minor source of cell-free DNA in a sample may be beneficial in estimating the level of certainty or power in detecting a copy number variation. Thus, the invention is intended to encompass both mechanisms for detection of SNP-containing genomic regions for direct determination of copy number variation through quantification as well as detection of SNP for ensuring overall efficiency of the assay.

Thus, in a particular aspect of the invention, allele-discrimination is provided through one of the first or second fixed sequence oligonucleotides. In this aspect, the first or second fixed sequence oligonucleotide is encompasses a SNP. In this aspect, the polymorphism is preferably located close enough to one end of as the fixed sequence oligonucleotide to provide allele-specificity through the ligation reaction. That is, in order to make the ligation allele-specific, the allele specifying nucleotide must be close to the ligated end. Typically, the allele-specific nucleotide must be within 5 nucleotides of the ligated end. In a preferred aspect, the allele-specific nucleotide is the penultimate or terminal base.

In certain aspects, allele detection results from the sequencing of a locus index or an allele index which is provided in one or both of the fixed sequence genomic region oligonucleotides. The locus index and/or allele index is embedded in either the first or second fixed sequence oligonucleotide used in the set for the selected genomic region containing a polymorphism, and is used with the specific fixed sequence oligo that is designed to detect the polymorphism. In this way, detection of the locus index and/or the allele index in an amplification product allows detection of the presence, amount or absence of the specific allele present in a sample, as well as the number of counts for the genomic region through quantification of the polymorphic products from the selected regions in the sample. In certain embodiments, the locus index and/or allele index is provided in the same fixed sequence oligonucleotide that encompasses the SNP. In other certain embodiments, the locus and/or allele index is provided in the other fixed sequence oligonucleotide in the set.

In specific aspects, an allele index is present on both the first and second fixed sequence oligonucleotides to detect two or more polymorphisms within the fixed sequence regions. The number of fixed sequence oligonucleotides used in such aspects correspond to the number of possible alleles being assessed for a selected genomic region, and sequence determination or hybridization of the allele index can detect presence, amount or absence of specific alleles and combinations of alleles in a sample.

For example, in one aspect of the invention, two or more separate reactions are carried out using a single locus and/or allele index and different fixed sequence oligonucleotides corresponding to the different polymorphisms in the selected genomic regions. The reactions are differentiated by the fixed sequence oligonucleotide, and the ligation, amplification and detection reactions comprising the different fixed sequence oligonucleotides remain separate through the detection step. The total counts for a particular genomic region of interest can be determined mathematically using the chromosomal index by determining the relative frequency of the genomic region from the separate reactions.

This aspect may be useful for, e.g., circumstances in which both information on polymorphic frequency in a sample and information on total loci counts are desirable. Since the reactions are detected separately, only one index may be needed for detection in each of the separate reactions.

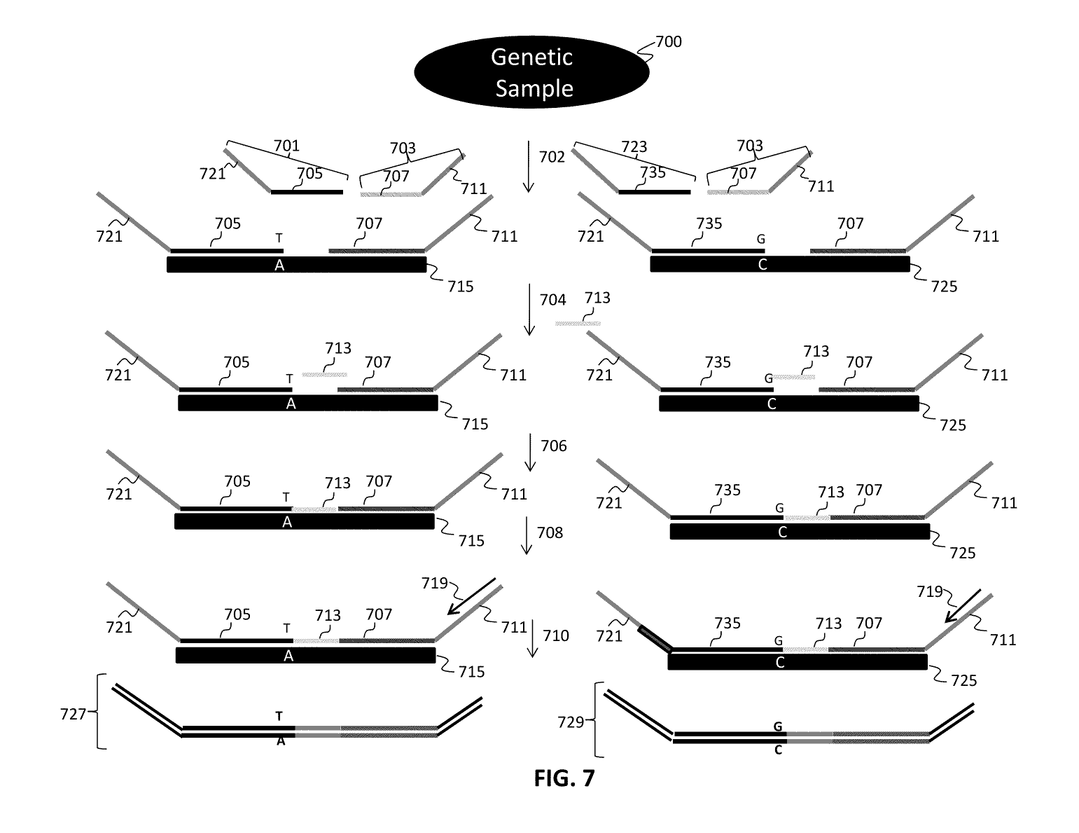

FIG. 7 illustrates this aspect of the invention. In FIG. 7, three fixed sequence oligonucleotides 701, 703 and 723 are used. Two of the fixed sequence oligonucleotides 701, 723 are allele-specific, comprising a region complementary to an allele in a genomic region comprising for example an A/T or G/C SNP, respectively. Each of fixed allele-specific oligonucleotides 701, 723 also comprises a corresponding allele index 721, 731. The second fixed sequence oligonucleotide 703 has a universal primer sequence 711, and this universal primer sequence is used to amplify the ligated oligonucleotides following initial selection and/or isolation of the selected genomic regions and the hybridized oligonucleotides in the sample. The universal primer sequence is located at the ends of the fixed sequence oligonucleotides 703 flanking the genomic regions of interest, and thus preserves the nucleic acid-specific sequences and the indices in the products of any universal amplification methods.

The fixed sequence oligonucleotides 701, 703, 723 are introduced 702 to the DNA sample 700 and allowed to specifically bind to the selected genomic region 715, 725. Following hybridization, the unhybridized fixed sequence oligonucleotides are preferably separated from the remainder of the sample (not shown). Bridging oligonucleotides 713 are introduced and allowed to hybridize 704 to the selected genomic region 715 between the first allele-specific fixed sequence oligonucleotide 701 and the second fixed sequence oligonucleotide 703 or to the selected genomic region 725 between the second allele-specific fixed sequence oligonucleotide region 723 and the second fixed sequence oligonucleotide region 703. Alternatively, the bridging oligonucleotides 713 can be introduced to the sample simultaneously with the sets of fixed sequence oligonucleotides.

The hybridized oligonucleotides are ligated 706 to create a ligation product spanning and complementary to the genomic regions of interest. The ligation primarily occurs only when appropriate allele-specific fixed sequence oligonucleotide is hybridized to the selected genomic region. Following ligation, universal primer 719 is introduced to amplify 708 the ligated products to create 710 amplification products 727, 729 that comprise the sequence of the genomic region of interest representing both SNPs in the selected genomic region. These amplification products 727, 729 are detected and quantified through digital PCR detection of the allele indices.

In some aspects, relative frequency information for a normal population is determined from normal samples that have a similar percent of DNA from a minor source. For example, an expected chromosomal dosage for trisomy in a DNA sample with a specific percent DNA from a minor source can be calculated by adding the percent contribution from the aneuploid chromosome. The relative frequency for the sample may then be compared to the relative frequency for a normal minor source in a sample and to an expected relative frequency if triploid to determine statistically, using the variation of the relative frequency, if the sample is more likely normal or triploid, and the value probability that it is one or the other.

While this invention is satisfied by aspects in many different forms, as described in detail in connection with preferred aspects of the invention, it is understood that the present disclosure is to be considered as exemplary of the principles of the invention and is not intended to limit the invention to the specific aspects illustrated and described herein. Numerous variations may be made by persons skilled in the art without departure from the spirit of the invention. The scope of the invention will be measured by the appended claims and their equivalents. The abstract and the title are not to be construed as limiting the scope of the present invention, as their purpose is to enable the appropriate authorities, as well as the general public, to quickly determine the general nature of the invention. In the claims that follow, unless the term “means” is used, none of the features or elements recited therein should be construed as means-plus-function limitations pursuant to 35 U.S.C. §112, ø16.

We claim:

1. A method for determining a frequency of genomic regions of interest in a sample, comprising the steps of:
providing a sample comprising a major and a minor source of cell-free DNA;
introducing at least two sets of first and second fixed sequence oligonucleotides to the sample under conditions that allow each set of fixed sequence oligonucleotides to specifically hybridize to different genomic regions of interest;
performing a ligation step to create ligation products;
amplifying the ligation products to create amplification products that reflect the relative frequency of the genomic regions of interest in the sample;
partitioning the amplification products into a plurality of discrete test sites such that the plurality of discrete test sites comprises either one or zero of the amplification products; and
analyzing the amplification products in the plurality of discrete test sites to provide a representation of the frequency of the genomic regions of interest in the sample.

2. The method of claim 1, further comprising extending the region between the first and second oligonucleotides of the sets of fixed sequence oligonucleotides with a polymerase and dNTPs to create adjacently hybridized fixed sequence oligonucleotides before performing the ligation step.

3. The method of claim 1, wherein the ligation product from a genomic region of interest is known to correspond to a genomic region of interest.

4. The method of claim 3, wherein the first and second genomic regions of interest are located on different chromosomes.

5. The method of claim 1, wherein at least one of the first and second fixed sequence oligonucleotides of the sets comprises a universal primer region.

6. The method of claim 1, wherein at least one of the first and second fixed sequence oligonucleotides of the sets comprises a chromosomal index.

7. The method of claim 6, wherein more than one fixed sequence oligonucleotides from selected genomic regions of the same chromosome have the same chromosomal index.

8. The method of claim 1, wherein at least one of the first and second fixed sequence oligonucleotides of the sets comprises a locus index.

9. The method of claim 1, wherein at least one of the first and second fixed sequence oligonucleotides of the sets comprises an allele index.

10. The method of claim 1, wherein the sample is a maternal sample comprising maternal and fetal DNA.

11. The method of claim 1, wherein the sample comprises cell-free DNA from a patient that has received a non-autologous transplant.

12. The method of claim 1, further comprising isolating the major and minor source cell-free DNA from the sample before introducing the at least two sets of first and second fixed

13. The method of claim 1, further comprising introducing one or more bridging oligonucleotides for each set of fixed sequence oligonucleotides under conditions that allow the bridging oligonucleotides to specifically hybridize to complementary regions in the genomic regions of interest between the fixed sequence oligonucleotides.

14. The method of claim 13, wherein the first and second fixed sequence oligonucleotides are introduced prior to introduction of the one or more bridging oligonucleotides.

15. The method of claim 13, wherein the one or more bridging oligonucleotides are introduced simultaneously with the at least two sets of first and second fixed sequence oligonucleotides.

16. The method of claim 1, further comprising determining the presence or absence of a copy number variation in the sample.

17. The method of claim 1, further comprising determining a value of probability of a copy number variation in the sample.

18. The method of claim 5, wherein the amplification utilizes primers comprising regions complementary to the universal primer sequences.

19. The method of claim 6, wherein analyzing the amplification products comprises introducing primers that are fluorescently labeled.

20. The method of claim 18, wherein the primers are added to the amplification products before partitioning the amplification products.

21. The method of claim 1, wherein analyzing the amplification products comprises detecting a presence or absence of a fluorescently labeled products corresponding to a genomic region of interest.

22. The method of claim 1, wherein analyzing the amplification products comprises performing a first detection reaction on a first set of genomic regions of interest and performing a second detection reaction on a second set of genomic regions of interest.

23. The method of claim 22, wherein the first set of genomic regions of interest are disposed on a first and second chromosome of interest.

24. The method of claim 23, wherein the amplification products corresponding to genomic regions on the first chromosome comprise a first chromosomal index and the amplification products corresponding to genomic regions on the second chromosome comprise a second chromosomal index.

25. The method of claim 24, wherein for a first detection reaction the amplification products corresponding to genomic regions on the first chromosome of interest comprise a first chromosomal index and amplification products corresponding to genomic regions on the second chromosome comprise a second chromosomal index and for a second detection reaction the amplification products corresponding to genomic regions on the first chromosome comprise the second chromosomal index and the amplification products corresponding to genomic regions on the second chromosome comprise the first chromosome index.

26. The method of claim 22, wherein the first detection reaction and the second detection reaction are performed simultaneously.

27. The method of claim 22, wherein the second detection reaction is performed after the first detection reaction.

28. A method for determining a frequency of genomic regions of interest in a sample, comprising the steps of:
providing a sample comprising a major and a minor source of cell-free DNA;
introducing at least two sets of first and second fixed sequence oligonucleotides to the sample under conditions that allow each set of fixed sequence oligonucleotides to specifically hybridize to different genomic regions of interest;
introducing one or more bridging oligonucleotides for each set of fixed sequence oligonucleotides under conditions that allow the bridging oligonucleotides to specifically hybridize to complementary regions in the genomic regions of interest, wherein the one or more bridging oligonucleotide is complementary to a region between the first and second fixed sequence oligonucleotides of the sets;
performing a ligation step to create continuous ligation products;
amplifying the continuous ligation products to create amplification products that reflect the relative frequency of the genomic regions of interest in the sample;
partitioning the amplification products into a plurality of discrete test sites such that the plurality of discrete test sites comprises either one or zero of the amplification products; and
analyzing the amplification products in the plurality of discrete test sites to provide a representation of the frequency of genomic regions of interest in the sample.

29. The method of claim 28, wherein the at least one bridging oligonucleotide hybridizes adjacent to the first or the second fixed sequence oligonucleotides of the sets.

30. The method of claim 28, wherein the at least one bridging oligonucleotides hybridizes adjacent to both the first and the second fixed sequence oligonucleotides of the sets.

31. The method of claim 28, wherein the at least one bridging oligonucleotide hybridizes to a complementary region in the genomic regions of interest such that the at least one bridging oligonucleotide is not adjacent to the first or second fixed sequence oligonucleotides of the set.

32. The method of claim 31, further comprising extending the region between the at least one bridging oligonucleotide and a non-adjacent fixed oligonucleotide with a polymerase and dNTPs to create adjacently hybridized fixed sequence oligonucleotides before performing a ligation step.

33. The method of claim 28, wherein at least one of the first and second fixed sequence oligonucleotides of the sets comprises a universal primer region.

34. The method of claim 28, wherein at least one of the first and second fixed sequence oligonucleotides of the sets comprises a chromosomal index.

35. The method of claim 4, wherein more than one fixed sequence oligonucleotides from selected genomic regions of the same chromosome have the same chromosomal index.

36. The method of claim 28, wherein at least one of the first and second fixed sequence oligonucleotides of the sets comprises a locus index.

37. The method of claim 28, wherein at least one of the first and second fixed sequence oligonucleotides of the sets comprises an allele index.

38. The method of claim 28, wherein the sample is a maternal sample comprising maternal and fetal DNA.

39. The method of claim 28, wherein the sample comprises cell-free DNA from a patient that has received a non-autologous transplant.

40. The method of claim 28, further comprising isolating the major and minor source cell-free DNA from the sample before introducing the at least two sets of first and second fixed sequence oligonucleotides.

41. The method of claim 28, further comprising determining the presence or absence of a copy number variation in the sample.

42. The method of claim 28, further comprising determining a value of probability of a copy number variation in the sample.

43. The method of claim 43, wherein the amplification utilizes primers comprising regions complementary to the universal primer sequences.

44. The method of claim 34, wherein analyzing the amplification products comprises introducing primers that are fluorescently labeled.

45. The method of claim 28, wherein analyzing the amplification products in the plurality of discrete test sites comprises detecting a presence or absence of fluorescently labeled products corresponding to a genomic region of interest.

46. The method of claim 28, wherein analyzing the amplification products comprises performing a first detection reaction on a first set of genomic regions of interest and performing a second detection reaction on a second set of genomic regions of interest.

47. The method of claim 46, wherein the first set of genomic regions of interest are disposed on a chromosome of interest and a reference chromosome.

48. The method of claim 47, wherein genomic regions of interest on the chromosome of interest comprise a first chromosomal index and nucleic acid regions of interest on the reference chromosome comprise a second chromosomal index.

49. The method of claim 48, wherein for the first detection reaction genomic regions of interest on the chromosome of interest comprise a first chromosomal index and nucleic acid regions of interest on the reference chromosome comprise a second chromosomal index and for the second detection reaction genomic regions of interest on the chromosome of interest comprise the second chromosomal index and nucleic acid regions of interest on the reference chromosome comprise the first chromosome index.

50. The method of claim 46, wherein the first detection reaction and the second detection reaction are performed simultaneously.

51. The method of claim 46, wherein the second detection reaction is performed after the first detection reaction.

52. A method for determining a frequency of genomic regions of interest in a sample, comprising the steps of:
providing a sample comprising a major and a minor source of cell-free DNA;
introducing at least two sets of first and second fixed sequence oligonucleotides to the sample under conditions that allow each set of fixed sequence oligonucleotides to different genomic regions of interest;
extending the region between the first and second oligonucleotides of the sets of fixed sequence oligonucleotides with a polymerase and dNTPs to create adjacently hybridized fixed sequence oligonucleotides;
performing a ligation step to create continuous ligation products;
amplifying the continuous ligation products to create amplification products that reflect the relative frequency of the genomic regions of interest in the sample;
partitioning the amplification products into a plurality of discrete test sites such that the plurality of discrete test sites comprises either one or zero of the amplification products; and
analyzing the amplification products in the plurality of discrete test sites to provide a representation of the frequency of genomic regions of interest in the sample.

53. The method of claim 52, wherein the ligation product from a genomic region of interest is known to correspond to a genomic region of interest.

54. The method of claim 52, wherein at least one of the first and second fixed sequence oligonucleotides of the sets comprises a universal primer region.

55. The method of claim 52, wherein at least one of the first and second fixed sequence oligonucleotides of the sets comprises a chromosomal index.

56. The method of claim 55, wherein more than one fixed sequence oligonucleotides from selected genomic regions of the same chromosome have the same chromosomal index.

57. The method of claim 52, wherein at least one of the first and second fixed sequence oligonucleotides of the sets comprises a locus index.

58. The method of claim 52, wherein at least one of the first and second fixed sequence oligonucleotides of the sets comprises an allele index.

59. The method of claim 58, wherein the ligation products from a genomic region of interest are known to correspond to a genomic region of interest.

60. The method of claim 52, wherein the sample is a maternal sample comprising maternal and fetal DNA.

61. The method of claim 52, wherein the sample comprises cell-free DNA from a patient that has received a non-autologous transplant.

62. The method of claim 52, further comprising isolating the major and minor source cell-free DNA from the sample before introducing the at least two sets of first and second fixed sequence oligonucleotides.

63. The method of claim 52, further comprising determining the presence or absence of a copy number variation in the sample.

64. The method of claim 52, further comprising determining a value of probability of a copy number variation in the sample.

65. The method of claim 54, wherein the amplification utilizes primers comprising regions complementary to the universal primer sequences.

66. The method of claim 55, wherein analyzing the amplification products comprises introducing primers that are fluorescently labeled.

67. The method of claim 66, wherein analyzing the amplification products in the plurality of discrete test sites comprises detecting a presence or absence of fluorescently labeled products corresponding to a nucleic region of interest.

68. The method of claim 52, wherein analyzing the amplification products comprises performing a first detection reaction on a first set of genomic regions of interest and performing a second detection reaction on a second set of genomic regions of interest.

69. The method of claim 68, wherein the first set of genomic regions of interest are disposed on a chromosome of interest and a reference chromosome.

70. The method of claim 69, wherein genomic regions of interest on the chromosome of interest comprise a first chromosomal index and nucleic acid regions of interest on the reference chromosome comprise a second chromosomal index.

71. The method of claim 70, wherein for the first detection reaction genomic regions of interest on the chromosome of interest comprise a first chromosomal index and nucleic acid regions of interest on the reference chromosome comprise a second chromosomal index and for the second detection reaction genomic regions of interest on the chromosome of interest comprise the second chromosomal index and nucleic acid regions of interest on the reference chromosome comprise the first chromosome index.

72. The method of claim 69, wherein the first detection reaction and the second detection reaction are performed simultaneously.

73. The method of claim 69, wherein the second detection reaction is performed after the first detection reaction.
